(19) United States
(12) Patent Application Publication
(10) Pub. No.: US 2021/0023318 A1
(43) Pub. Date: Jan. 28, 2021

(54) Nasal Airway Pressure Monitoring System

(71) Applicant: Dan Tatum, Brookhaven, PA (US)

(72) Inventor: Dan Tatum, Brookhaven, PA (US)

(21) Appl. No.: 16/523,665

(22) Filed: Jul. 26, 2019

(51) Int. Cl. A61M 16/00; A61M 16/06; A61M 16/16; A61M 16/10

(52) U.S. Cl. CPC A61M 16/024; A61M 2205/581; A61M 16/16; A61M 16/101; A61M 16/0069; A61M 16/106; A61M 16/0051; A61M 16/0003; A61M 2016/0027; A61M 2205/50; A61M 2016/0033; A61M 2240/00; A61M 2016/1025; A61M 2205/587; A61M 16/0672

(57) ABSTRACT

A nasal airway pressure monitoring system and method for use with neonatal patients is provided. The nasal airway pressure monitoring system has a nasal catheter, a pressure monitor, and a respiratory circuit supplying a respiratory gas. The nasal catheter is connected at a first end to the pressure monitor via a sample line and the second end of the nasal catheter is positioned in a nares. Upon delivery of gas flow through the patient respiratory circuit, the nasal catheter transmits pressure waveform data from the nasal airway to the pressure monitor. The pressure monitor processes the pressure waveform data. If the mean airway pressure is outside of a pre-set range of maximum and minimum limits, an alarm is triggered.

### FIELD OF THE INVENTION

The invention relates to a nasal airway pressure monitoring and alarm system and, more particularly, to a neonatal nasal airway pressure monitoring and alarm system. The invention is useful to monitor and adjust airway pressure, preferably in a neonatal patient.

### BACKGROUND

Infants with immature respiratory systems or other conditions which impair lung function use the resources of neonatal intensive care units (NICU) to monitor and alert practitioners to untherapeutic airway pressure. Ventilation equipment provides pressurized gases to the patient, a procedure that can introduce injury to lung tissue in the instance of over-pressurization. Under-pressurization can deprive the patient of therapeutic oxygen levels and produce hypoxia. An inability to measure and adjust airway pressure to a therapeutic level is a long-standing challenge that hampers a practitioner's ability to provide a continuous safe level of pressurized airway gas. “Practitioners” refer to individuals operating the claimed invention and include a physician, a respiratory therapist, a nurse, or other medical personnel.

There are disadvantages to known equipment which restrict the ability of practitioners to beneficially monitor and adjust the patient's nasal airway pressure. Respiratory ventilation equipment include nasal Continuous Positive Airway Pressure (nCPAP) and humidified high flow therapy (HFT). The nCPAP technology is widely used because it can be administered non-invasively to allow a patient's air sacs to open for improved oxygenation. The nCPAP, however, presents disadvantages including fitting of the equipment to avoid physical distress. The HFT systems provide greater relative comfort, but presents challenges in monitoring and adjusting airway pressures to therapeutic levels.

A lack of information regarding the pressure of respiratory gases within the patient's airway is an obstacle to maintaining continuous airway gas at a therapeutic level. Accurate and timely measurement and adjustment of the airway gas pressure is useful to treat effects of under- or over-pressurization within the airway.

Innovative tools and methods for addressing the needs of neonatal patients are therefore of significant interest to practitioners. Bearing in mind the deficiencies of the existing technology, a nasal airway pressure monitoring system has been developed that provides technical features not previously foreseen.

### SUMMARY

The claimed invention overcomes problems of the known art and provides an improved neonatal pressure monitoring and alarm system that monitors the mean airway pressure in nares to sustain a continuous positive nasal pharyngeal pressure. The claimed invention is particularly useful in the treatment of neonatal patients.

The present invention does not measure gas pressure from within a respiratory circuit tubing that delivers respiratory gas to a patient airway. Instead, in the claimed invention the pressure monitor receives waveform data through a nasal catheter placed distal to and outside of a nasal cannula supplying pressurized respiratory gas into the nasal cavity. The claimed invention is operable in both open and closed systems. The claimed invention allows a practitioner to adjust mean airway pressure within pre-set minimum and maximum settings. The alarm element operates when the mean airway pressure falls outside of the pre-set minimum and maximum limits. The claimed invention is not limited to use with any particular respiratory circuit or nasal cannula.

The preceding paragraphs are provided as an introduction and are do not limit the scope of the claimed invention. The provided embodiments, along with advantages of the invention, are appreciated by reference to the following detailed description and with the accompanying drawings.

### BRIEF DESCRIPTION OF THE DRAWINGS

The invention will be explained in greater detail below with reference to embodiments, referring to the appended Figures, in which:

FIG. 1 is a schematic diagram illustrative of a neonatal pressure alarm monitoring system according to a first embodiment of the invention using a one way flow of high flow (HF) pressurized respiratory gas.

FIG. 2 is a schematic diagram illustrative of a neonatal pressure alarm monitoring system according to a second embodiment of the invention using a bubble respiratory circuit (nCPAP).

FIG. 3 is an illustration of a nasal catheter according to an embodiment of the invention.

FIG. 4 is a flow chart of a method of monitoring and adjusting naral airway pressure according to one embodiment of the invention.

### DETAILED DESCRIPTION OF THE EMBODIMENT(S)

Exemplary embodiments of the present invention will be described hereinafter in detail with reference to the attached drawings, wherein like reference numerals indicate identical or corresponding elements throughout several figures. The present invention may, however, be embodied in many forms and should not be construed as being limited to the embodiments set forth herein. Rather, these embodiments are provided to convey the concept of the disclosure to those skilled in the art.

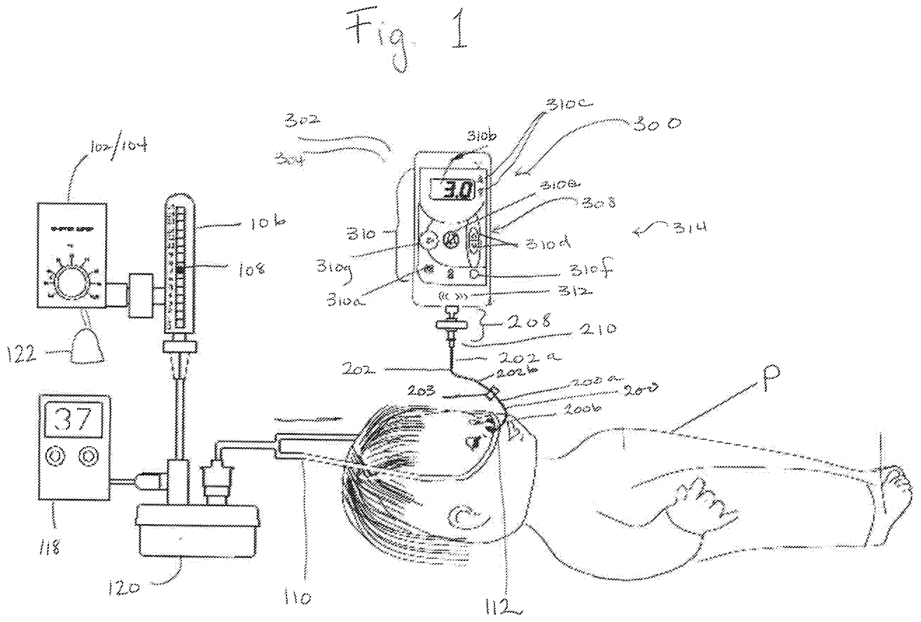

The following description refers to a nasal airway pressure monitoring system 1 and a method for monitoring, alerting, and adjusting airway mean gas pressure, preferably useful in neonatal patients P. FIGS. 1-3 show a nasal airway pressure monitoring system 1 according to the invention. In various embodiments of the invention, a nasal catheter is positioned to measure gas pressure within the nasal cavity.

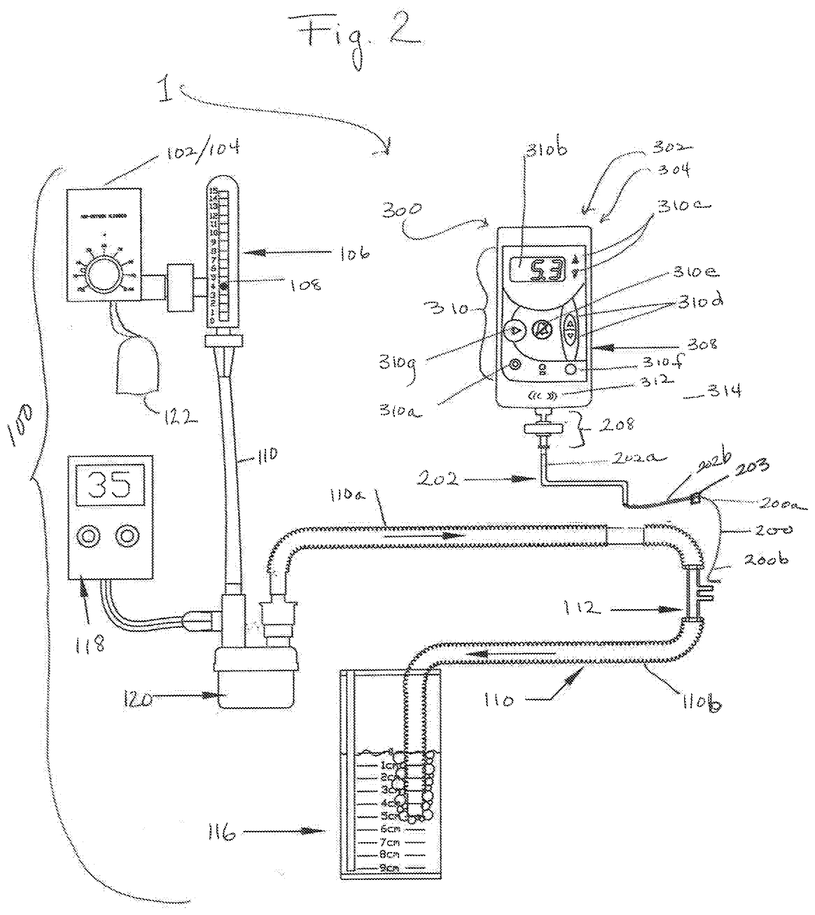

FIGS. 1 and 2 are schematic diagrams illustrative of the claimed nasal pressure monitoring system 1. The claimed invention is comprised of a respiratory circuit 100, a nasal catheter 200, and a pressure monitor 300. FIG. 1 illustrates a first embodiment of the invention using a one way flow of pressurized respiratory gas supplied to the patient's airway. The respiratory circuit 110 allows respiratory gas to escape directly to the atmosphere from the nasal cavity upon the patient's exhalation. FIG. 2 illustrates a second embodiment of the invention using a bubble CPAP respiratory circuit 110 in which pressurized respiratory gas is supplied via a first section 110a through a nasal cannula 112 to the nasal cavity and then is directed upon exhalation through a second section 110b via a “bubble” canister 116 before being released to the atmosphere.

Respiratory Circuit

The respiratory circuit 100 comprises CPAP equipment chosen by one of ordinary skill in the art to address the respiratory needs of the patient. The respiratory circuit 100 is comprised of a source of respiratory gas 122, a flowmeter 106 including a set of controls 108 to adjust the rate of respiratory gas flowing to the patient and for setting the flow rate of respiratory gas; and a circuit of tubing 110 including a nasal cannula 112 for directing the respiratory gas to the naso-pharyngeal cavity of the patient. The respiratory circuit 100 may further include a blower 102 to propel respiratory gas through the circuit, an air and oxygen blender 104 to create a desirable composition of respiratory gas(es), a tubing circuit 110, a nasal cannula 112. The respiratory circuit 100 is connected to a power source 114. Another embodiment of the invention further comprises a bubble CPAP canister 116 as part of the respiratory circuit 100. A source of respiratory gas 122 is connected to the respiratory circuit 100. The respiratory circuit 100 includes medical grade tubing 110 through which pressurized respiratory gas 122 is supplied to the patient's nares through a nasal cannula 112. Where the nares are not sealed, pressurized respiratory gas 122 can escape directly to the atmosphere. The CPAP equipment may also include a humidifier 120, an oxygen analyzer 118, a bubble CPAP canister 116, and other accessories. These elements are arranged to deliver pressurized respiratory gas to the nasal cavity of the patient P.

Nasal Catheter

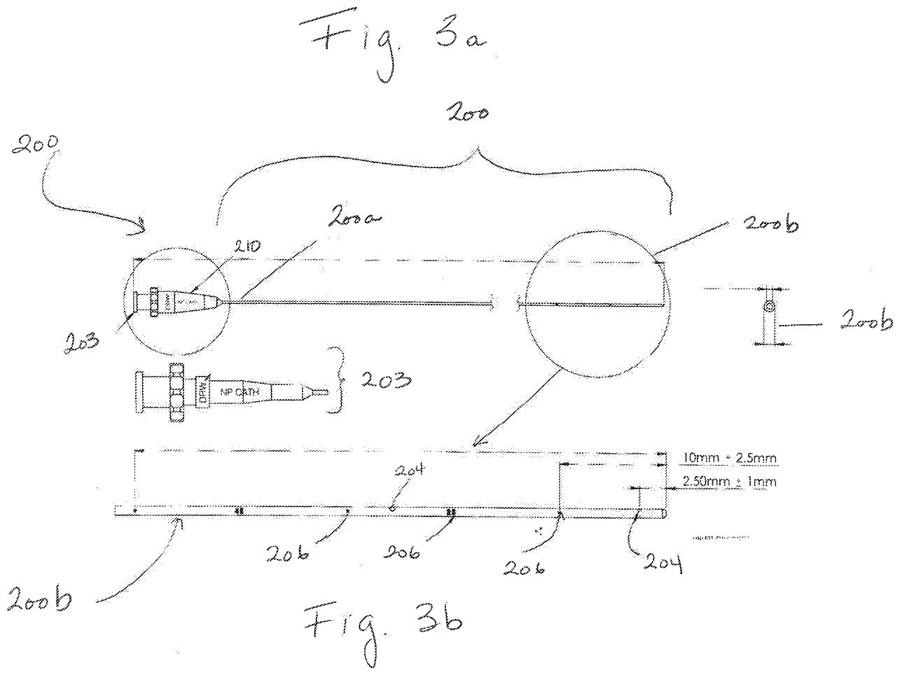

The claimed invention additionally comprises a nasal catheter 200. The nasal catheter 200 transmits a pressure waveform obtained from the patient's nares to a pressure monitor 300 having a processor 304 as described below. FIG. 3 is an illustration of a nasal catheter 200 according to an embodiment of the invention. A suitable nasal catheter 200 for use with various embodiments of the invention include DRW Nasal Catheter (DRW Medical LLC, DRW Medical LLC, 811 Lincoln Dr., Brookhaven, Pa. 19015).

The nasal catheter 200 is preferably composed of a cylindrical non-radiopaque medical grade tubing which may be polyurethane or 2.5, 3.5 and 4.3 Fr Pellethane. The choice of tubing reflects the needs of a particular patient, but the generally preferred size is 2.5. A sample line 202 is joined via a first luer attachment 208 at a first end 202a to the pressure monitor 300. The sample line 202 contains a filter (not visible). The sample line 202 is connected on a second end 202b via a second luer attachment 203 to the nasal catheter 200 at a first end 200a. A second end 200b of nasal catheter 200 is placed in a nares of the patient P. A suitable length of both the sample line 202 and the nasal catheter 200 is about 300 mm. Cross-sectional dimensions of the nasal catheter 200 are preferably an outer dimension of 0.82 mm (0.032″) and an inner dimension of 0.43 mm (0.017″). Other suitable dimensions are known to those of ordinary skill in the art and are chosen with regard to the needs of the patient. The second end 200b of the nasal catheter 200 is open to the interior of the cylindrical tubing and is smoothly rounded without a surface or edge that could cause injury to the patient P upon insertion into the nasal cavity.

A further embodiment of the nasal catheter 200 includes a portal(s) 204 preferably located between about 2.5 mm and about 5 mm from the second end 202b of the nasal catheter 200. The portal 204 is a generally cylindrical passage about 0.33 mm (0.013″) in diameter, runs between the interior and the exterior of the catheter, and serves to prevent occlusion of the nasal catheter 200 by mucus or water that may be present in the nasal cavity. A placement gauge 206 is marked in 10 mm intervals in one embodiment on the exterior of the nasal catheter as measured from the second end 200b. The placement gauge 206 is used to position the second end 200b of the nasal catheter 200 within the nares.

Pressure Monitor:

A preferred pressure monitor 300 for use with various embodiments of the invention includes DRW NAPA LP-15 Airway Pressure Monitor (e.g., DRW Medical LLC, 811 Lincoln Dr., Brookhaven, Pa. 19015). The pressure monitor 300 comprises a pressure sensor 302 and processor 304 that measures the mean airway pressure transmitted via the nasal catheter 200 to which it is connected. It measures pressures in both open and closed systems. The DRW NAPA LP-15 Airway Pressure Monitor measures positive and negative pressure airway pressure over complete breathing cycles (inhalation and expiration) and registers a mean airway pressure. It measures atmospheric pressure as zero.

Alarms 306 (audible 306a and visual 306b) of the pressure monitor 300 are triggered when the pressure sensor 302 detects airway pressure more than 0.1 cm H₂O above a pre-set maximum limit or more than 0.1 cm H₂O below a pre-set minimum limit. The high pressure alarm limit can be set from 6.0 to 35.0 cm H₂O. The low pressure limit can be set from 0.1 to 28.0 cm H₂O. The accuracy of the pressure sensor 302 is +/−0.5 cm H₂O or +/−2%, whichever is greater. Additionally, disconnection of the nasal catheter 200 from the pressure monitor 300 triggers the audible and visual alarms 306a, 306b. The alarms 306 reset when the desired airway pressure is restored.

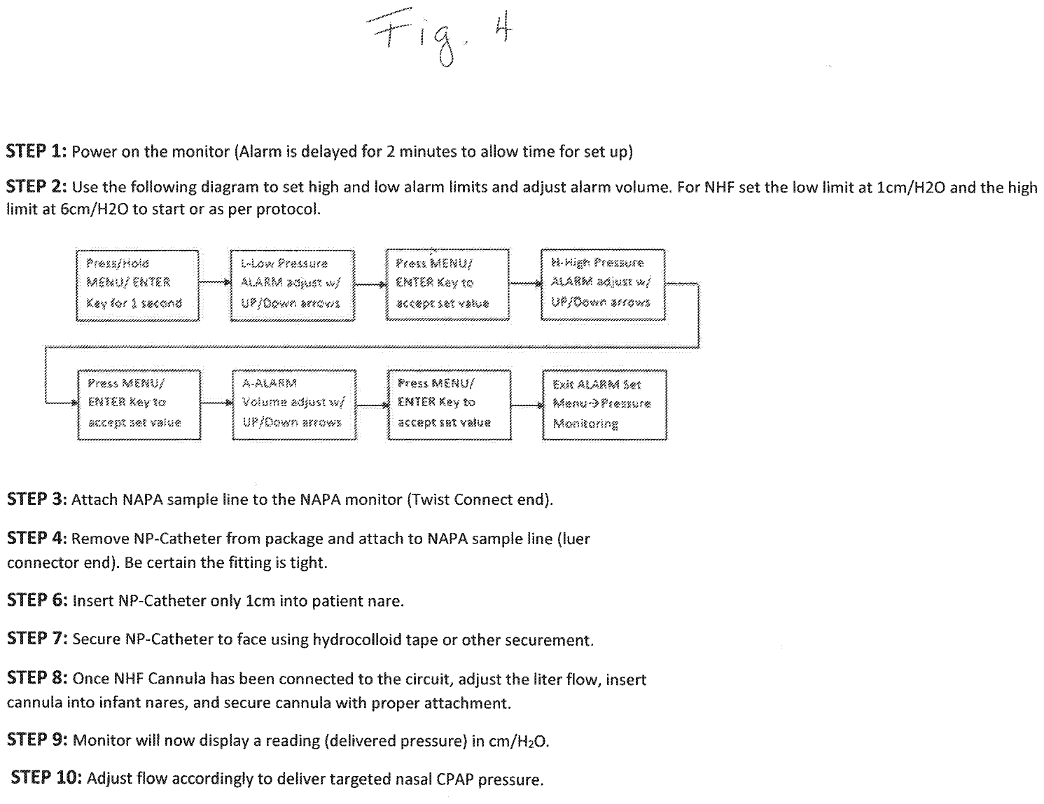

The pressure monitor 300 comprises a housing 308 having control panel 310 for operating and adjusting the features of the pressure monitor 300. FIG. 4 illustrates one embodiment of the control panel 300. The control panel 300 includes an on-off switch 310a, a pressure display 310b to visually indicate the mean airway pressure of the patient, preferably displayed with LED lights. The control panel 300 includes a menu key 310g to select and enter a chosen setting. The pressure monitor 300 has a speaker 312 and a control 310d (toggle controls) to select maximum pressure and a minimum pressure limits and to adjust audible alarm volume. The visual pressure indicators 310d blink upon pressure falling below or exceeding the pre-set limits and are preferably an LED or similar source of light. The pressure monitor 300 includes a control 310e to suspend the audible alarm 306a. The control panel 310 also includes a control 310f to register atmospheric pressure (non-pressurized) at zero.

Software for the NAPA LP-15 microprocessor is designed for measuring low pressures and used specifically for a neonatal patient. A BLVR pressure sensor is utilized and calibrated with a calibration device and software to provide an accurate pressure range and sensitivity offered by the device.

The sample line 202 is removably connected at the pressure monitor 300 at a first end 202a through a luer lock connection 208. A suitable choice for a luer lock connection 208 is a HUB-1 luer lock hub which conforms to ISO 594 for conical fittings. The luer lock connection 208 is preferably sheathed with a sleeve 210 of silicon or other material known to one of skill in the art.

The nasal airway pressure monitoring system 1 provides mean airway monitoring of airway pressure. The measured mean airway pressure data is used to adjust the flow rate of pressurized gas to meet patient requirements. Respiratory gases are supplied through nasal cannulas at flow rates of 1-10 liters per minute (L/min) to yield a desired targeted mean airway pressure. Standard treatment protocols use heated and humidified respiratory gases which allow practitioners to use flow rates at the higher end of the scale without irritation to the patient. Equipment for use in transport may not include heated and humidified respiratory gas flow. The claimed invention is suitable for use with either system.

High flow nasal airway respiratory support (“high flow therapy” or “HFT”) is administered through a nasal cannula into an “open” nasal airway, i.e., one that is not sealed or “closed”). The effects of such high flow therapies are reported as therapeutic and embraced by some clinicians while questioned by others because heretofore HFT involved unknown factors and arbitrary administration techniques. In such procedures, the pressures generated in the patients' airways are variable, affected by cannula size, nares size, flow rate, and breathing rate, for instance. It is known that airway pressures affect oxygen saturation, thus these variables are enough to keep many physicians from utilizing HFT to avoid over pressurization. The claimed invention is used by practitioners to monitor nasal pressure as follows and as illustrated in FIG. 4. FIG. 4 is a flow chart of the steps of the claimed method of monitoring and adjusting airway pressure according to an embodiment of the invention. The steps set out are applicable to the embodiment of the claimed invention and with reference to FIGS. 1-4.

Step 1: Power on the pressure monitor 300. The alarm 306 is delayed for a chosen number of minutes to allow time for system set up. Two minutes is a suitable choice.

Step 2: Set maximum and minimum alarm triggering limits and adjust the volume of the alarm to a desired level. Set the maximum limit at 8-10 cm/H₂O and the minimum limit at 1-4 cm/H₂O or as per patient-specific protocol therefore bracketing the desired targeted pressure with the high and low alarm settings.

Step 3: Attach the NAPA sample line 202 to the NAPA LP-15 pressure monitor 300.

Step 4: Attach the first end 200a the nasal catheter 200 it to the NAPA-15 sample line 202 at the luer lock connector 208, checking to ensure a secure and airtight fit on the patient.

Step 5: Insert the second end 200b of the nasal catheter 200 into the patient nares approx. 1-2 cm distally, measuring from the entrance of the nares.

Step 6: Secure the nasal catheter 200 to the face of the patient using a hydrocolloid tape T (not shown) or other appropriate securement.

Step 7: Once the nasal cannula 112 has been connected to the respiratory circuit 100, adjust the respiratory gas flow typically at 3-4 L to start using the flowmeter 106, insert the high flow nasal cannula 112 into the patient nares, and secure the nasal cannula 112 in place with hydrocolloid tape Y (not shown) or other appropriate material.

Step 8: The pressure monitor 300 will now display a reading of the delivered airway pressure in cm/H₂O.

Step 9: Adjust the gas flow using the flowmeter 106 to a flow that results in a desired positive airway pressure or dynamic CPAP delivered to the patient's airway. This may require a respiratory gas flow rate of up to 8 or 10 Liters per minute to obtain a desired targeted positive pressure in the patient airway.

The pressure monitor 300 provides an alarm 306 (aural, visual) upon determination that the mean airway pressure falls outside a pre-set minimum-maximum range.

The claimed method for monitoring mean airway pressure in neonatal patients comprises providing a patient with the neonatal nasal airway pressure system according to the nasal airway pressure monitoring system as claimed and described above; placing and securing a second end of a nasal catheter within the nares of the patient at about 1-2 cm distal to a nasal cannula; measuring the breathing cycle of the patient with the pressure monitor by acquiring a data set representative of a waveform obtained from the nasal catheter to establish a mean airway pressure; and adjusting the gas flow to establish a mean airway pressure typically within a range yielding 4-6 cm/H₂O.

The foregoing illustrates some of the possibilities for practicing the invention. Many other embodiments are possible within the scope and spirit of the invention. It is, therefore, intended that the foregoing description be regarded as illustrative rather than limiting, and that the scope of the invention is given by the appended claims together with their full range of equivalents.

What is claimed is:

1. A neonatal nasal airway pressure monitoring system comprising
i. a pressure monitor having
1. a pressure sensor;
2. a processor;
3. a control panel;
4. a measurement display; and
5. an alarm triggered by airway pressure outside of pre-set limits for maximum or minimum mean airway pressure;
ii. a nasal catheter for transmitting a pressure waveform to the pressure monitor, the nasal catheter comprising medical grade tubing and removably securable at a first end to a sample line of medical grade tubing connected to the pressure monitor at a second end and placeable at a second end within a nare of a patient; and
iii. a respiratory circuit comprising
1. a source of respiratory gas;
2. a flowmeter for setting the flow rate of respiratory gas; and
3. a circuit of tubing and a nasal cannula for directing the respiratory gas to the naso-pharyngeal cavity of the patient.

2. The neonatal nasal airway pressure system of claim 1 wherein the respiratory gas is supplied through the nasal cannula typically at a flow rate of approximately 1-10 liters per minute to obtain a desired mean targeted positive pressure in the patient airway.

3. The neonatal nasal airway pressure monitoring system of claim 1, the nasal catheter having an outer diameter of about 0.82 mm (0.032″) and an inner diameter of about 0.43 mm (0.017″).

4. The neonatal nasal airway pressure monitoring system of claim 1, wherein the second end of the nasal catheter has a portal between the interior and exterior of the nasal catheter and located between about 2.5 mm and about 5 mm proximal from the second end.

5. The neonatal nasal catheter according to claim 1, wherein the second end further comprises a series of placement gauge markings.

6. The neonatal nasal catheter according to claim 1, wherein the second end is rounded.

7. The neonatal nasal catheter according to claim 1, wherein the first end has a luer lock connector removably securable to the second end of the sample line.

8. The neonatal nasal catheter according to claim 1, wherein the first end further comprises a sleeve.

9. The neonatal nasal catheter according to claim 1, wherein the nasal catheter is non-radiopaque.

10. The neonatal nasal airway pressure monitoring system according to claim 1, wherein the respiratory circuit further comprises a humidifier.

11. The neonatal nasal airway pressure monitoring system according to claim 1, wherein the respiratory circuit further comprises an oxygen blender.

12. The neonatal nasal airway pressure monitoring system according to claim 1, wherein the respiratory circuit further comprises an oxygen analyzer.

13. A method for monitoring mean airway pressure in neonatal patients comprising
i. providing a patient with the neonatal nasal airway pressure system according to claim 1;
ii. placing a second end of a nasal catheter within a naral of a patient about 1-2 cm distal to a nasal cannula.
iii. measuring the breathing cycle of the patient by acquiring a data set representative of a waveform from the nasal catheter; and
iv. adjusting the flow rate of pressurized respiratory gas to yield the desired targeted mean airway pressure.
